(19) United States
(12) Patent Application Publication
(10) Pub. No.: US 2022/0117790 A1
(43) Pub. Date: Apr. 21, 2022

(54) WOUND DRESSING WITH SELECTIVE AND DYNAMIC TRANSPARENCY

(71) Applicant: KCI LICENSING, INC., San Antonio, TX (US)

(72) Inventors: Christopher B. LOCKE, Bournemouth (GB); Timothy M. ROBINSON, Wimbourne (GB)

(21) Appl. No.: 17/421,253

(22) PCT Filed: Jan. 9, 2020
(86) PCT No.: PCT/US2020/012889

§ 371 Date: Jul. 7, 2021

Related U.S. Application Data
(60) Provisional application No. 62/795,721, filed on Jan. 23, 2019.

(51) Int. Cl. A61F 13/00

(52) U.S. Cl. CPC A61F 13/00008; A61F 13/00059; A61F 13/00055; A61F 2013/00336; A61F 13/00029; A61F 2013/00182; A61F 13/00068

(57) ABSTRACT

Wound therapy dressings are provided. The wound therapy dressings may include at least one selectively transparent layer with a refractive index in a range wherein interaction between the selectively transparent layer and fluids from a treatment site switches the selectively transparent layer from a first, opaque state to a second, transparent state. Dressings that include multiple layers may include polymer films with a plurality of fluid restrictions, a manifold, a transparent polymer drape, and a hydrophobic polymer layer. The dressings may further include negative pressure therapy treatment input devices. A method of use wherein the dressings are used to monitor fluid flow, exudate, and maceration around a treatment site is also provided.

### CROSS-REFERENCE TO RELATED APPLICATIONS

This application claims the benefit of priority to U.S. Provisional Application No. 62/795,721, filed on Jan. 23, 2019, which is incorporated herein by reference in its entirety.

### FIELD

The invention set forth in the claims relates generally to tissue treatment and more particularly, but without limitation, to dressings for tissue treatment and methods for using the dressings for tissue treatment both with and without negative pressure.

### BACKGROUND

One of the risks in wound therapy is maceration, which occurs when a treatment site is exposed to moisture or fluids for an excessive period of time. Existing dressings are typically colored or structured in a way that makes seeing the location of exudate a challenge, unless the dressing is removed. Dressing removal can be both painful to the patient and unnecessarily disturb granulation. Seeing the presence and location of exudate in a wound bed without having to completely remove or change dressings would allow clinicians to more accurately assess the risk of maceration with the added benefit of leaving the wound bed undisturbed. It would therefore be desirable to provide a simple and optional means of identifying where fluid is in a treatment site and the direction of flow of the fluid, while maintaining the integrity and constitution of the therapy dressing.

### SUMMARY

The present disclosure overcomes the drawbacks of previously-known dressings by providing a selectively transparent wound therapy dressing and method of use. In one embodiment, the wound therapy dressing may include at least one selectively transparent layer. The selectively transparent layer may have a refractive index wherein interaction between the layer and fluid from a wound switches the layer from a first, opaque state to a second, transparent state. The layer also may extend over both a wound and a periwound. Fluid from a wound may include, but is not limited to, exudate or secretions from a treatment site. The selectively transparent layer further may include or have microporous particles. The selectively transparent layer also may be coated with microporous particles.

The refractive index of the selectively transparent layer may be within a range that is substantially similar to that of the fluid from a wound. In some embodiments, the selectively transparent layer may have a refractive index between about 1.0 and about 2.0. In some embodiments, the selectively transparent layer may have a refractive index between about 1.2 and about 1.75. In some embodiments, the selectively transparent layer may have a refractive index between about 1.33 and about 1.6.

The selectively transparent layer also may include or be made of minerals. Exemplary minerals include, but are not limited to, silicas, silicates, fluorites, or fluorides. The selectively transparent layer also may comprise polymers. Exemplary polymers include, but are not limited to, fluoropolymers, silicones, or acrylics.

A wound therapy dressing including four distinct layers that are configured to be assembled in a stacked relationship is also disclosed. The first layer may be a polymer film have a plurality of fluid restrictions. The first layer may be configured to be applied to a treatment site. In some embodiments, the second layer may be a manifold. The third layer may be a selectively transparent layer, wherein in a first state the selectively transparent layer is opaque when dry and in a second state the selectively transparent layer is translucent when wetted by a fluid with a refractive index substantially similar to the selectively transparent layer. The fourth layer may be a transparent polymer drape. The second state, wherein the selectively transparent layer is opaque, may occur at a point in time subsequent to the first state.

The fluid from a wound may be an exudate or a secretion from a treatment site. The first layer also may be configured to expand in response to a pressure gradient across the film. In some embodiments, the third layer is laminated on top of the second layer and underneath the fourth layer. The third layer also may be perforated. The fourth drape layer may extend over and adhere the first layer, the second layer, and the third layer to a wound treatment site.

In some embodiments, the selectively transparent layer may return to the first, opaque state as the fluid evaporates. Further, a first portion of the selectively transparent layer may become translucent when exposed to fluid, such as exudate, and any remaining portions of the selectively transparent layer not exposed to the fluid may remain opaque.

The dressing further may comprise a negative pressure wound therapy input or interface device. In some embodiments, the negative pressure wound therapy input device couples to a hole in the drape layer through which air may be withdrawn from the treatment site.

In some embodiments, the manifold may be a foam, including, but not limited to, a polyurethane foam. The foam also may be porous and have an average pore size in the range of 400-600 microns. The selectively transparent layer also may be comprised of or coated with microporous particles.

The refractive index of the selectively transparent third layer may be within a range that is substantially similar to that of the fluid from a wound. In some embodiments, the selectively transparent layer may have a refractive index between about 1.0 and about 2.0. In some embodiments, the selectively transparent layer may have a refractive index between about 1.2 and about 1.75. In some embodiments, the selectively transparent layer may have a refractive index between about 1.33 and about 1.6.

The selectively transparent third layer also may include or be made of minerals. Exemplary minerals include, but are not limited to, silicas, silicates, fluorites, or fluorides. The selectively transparent layer also may comprise polymers. Exemplary polymers include, but are not limited to, fluoropolymers, silicones, or acrylics.

A clear adhesive may be coated on the drape before the addition of microporous particles. The manifold also may be a corrugated structure. Further, the selectively transparent layer may be pre-perforated with a hole for the communication of pressures and fluids. The pre-perforation may be done by a user or may be manufactured with perforations prior to use.

The selectively transparent layer may indicate the presence or absence of fluids over the periwound of a wound bed. One or more of the manifold, the selectively transparent third layer and the microporous particles may be hydrochromic. Any hydrochromic layer may be polyvinylidene difluoride (PVDF). In one alternative embodiment, the selectively transparent layer may comprise areas in an alternating radial pattern extending from a center to an outside edge of the dressing.

A method of use for a wound therapy dressing is also provided herein. The method may include: i) applying to a treatment site a wound therapy dressing having at least one selectively transparent layer with a refractive index that switches the selectively transparent layer from a first, opaque state to a second, transparent state, when the layer interacts with fluid from a wound; (ii) sealing the dressing to epidermis adjacent or around the treatment site; and (iii) observing the selectively transparent layer for at least one transparent region, wherein a transparent region on the selectively transparent layer indicates fluid in that area of the treatment site. In certain embodiments of the method, the selectively transparent layer extends over both wound and periwound.

In the method above, the transparent region may indicate the presence of fluid treatment may prompt a caregiver to take action to avoid possible maceration and the method of wound therapy may be used to prevent maceration of a wound or periwound. The presence or absence of the transparent region may also indicate the moisture vapor transmission rate of the dressing. In certain embodiments of the method, the dressing further may comprise a negative pressure wound treatment input device. The negative pressure wound treatment input device may couple to a hole in the drape layer and air may be withdrawn from the treatment site through the hole. This method may be used to activate the negative pressure input device and use the pressure to promote healing and tissue granulation.

### BRIEF DESCRIPTION OF THE DRAWINGS

FIG. 1A shows a top view of an exemplary wound therapy dressing prior to interaction with a fluid from a wound.

FIG. 1B depicts a top view of a wound therapy dressing at 125 mmHg prior to fluid delivery.

FIG. 1C illustrates a top view of a wound therapy dressing wherein an initial fluid instillation is being delivered to the base of the wound and the selectively transparent layer has begun to switch from a first state to a second state.

FIG. 1D shows a top view of a wound therapy dressing where 60 mL of fluid has been delivered to the dressing, and the second, transparent state is visible.

FIG. 1E illustrates a top view of a wound therapy dressing where 120 mL of fluid has been delivered to both the wound and periwound areas and is beginning to be withdrawn.

FIG. 1F shows a top view of a wound therapy dressing where the selectively transparent layer has dried and is opaque. The moisture vapor transmission rate has dried the switching layer.

FIG. 2A depicts an exemplary wound therapy dressing of the present disclosure in exploded view before it is assembled.

FIG. 2B depicts an exemplary wound therapy dressing of the present disclosure in exploded view before it is assembled that includes a negative pressure wound treatment input device.

FIG. 3A depicts an exemplary wound therapy dressing in exploded view before it is assembled and in relation to a treatment site.

FIG. 3B depicts an exemplary wound therapy dressing in exploded view before it is assembled and in relation to a treatment site including a negative pressure wound treatment input device.

FIG. 4A depicts an alternative embodiment wherein the wound therapy dressing further includes a hydrophobic polymer.

FIG. 4B depicts an alternative embodiment wherein the wound therapy dressing further includes a hydrophobic polymer and a negative pressure wound treatment input device.

### DETAILED DESCRIPTION

### Wound Therapy Dressings:

The presently disclosed dressings and method may be used in a variety of proprietary negative pressure wound therapy systems, including, but not limited to those shown and described in U.S. patent application Ser. No. 15/997,761, filed on Jun. 5, 2018, the entire contents of which are hereby incorporated by referenced, V.A.C.® Therapy, V.A.C. VERAFLO™, TIELLE™ Dressing Family, KERRAFOAM™ Dressings, and PREVENA™ Therapy Platform. The dressings and method may also be used in conjunction with fluid state indicators for absorbent wound management systems, other wound dressings with selective and dynamic transparency, and fluid collection canisters with user selectable content visualization.

As used herein, the terms “substantially,” “substantial,” and “about” are used to describe and account for small variations. When used in conjunction with a material or a standard, the terms can refer to instances in which the standard or material occurs precisely as well as instances in which the event or circumstance occurs to a close approximation. For example, the terms can refer to less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

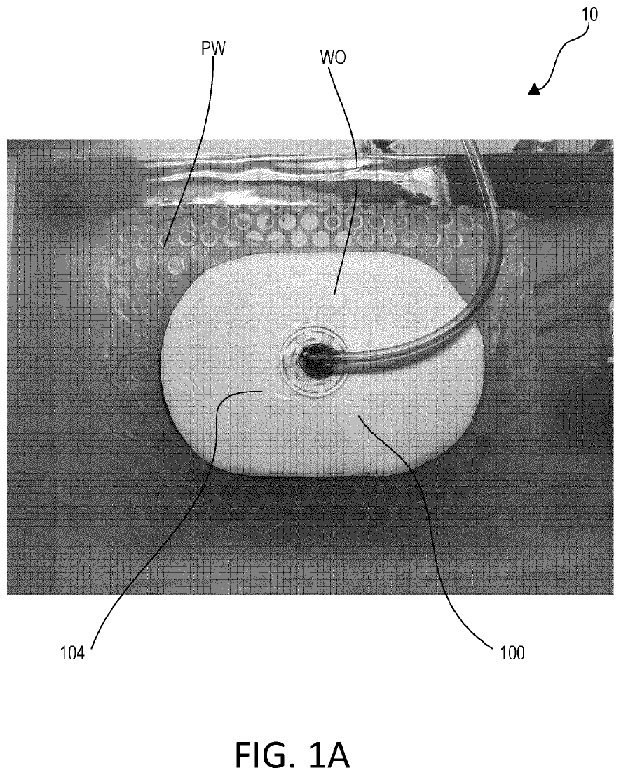

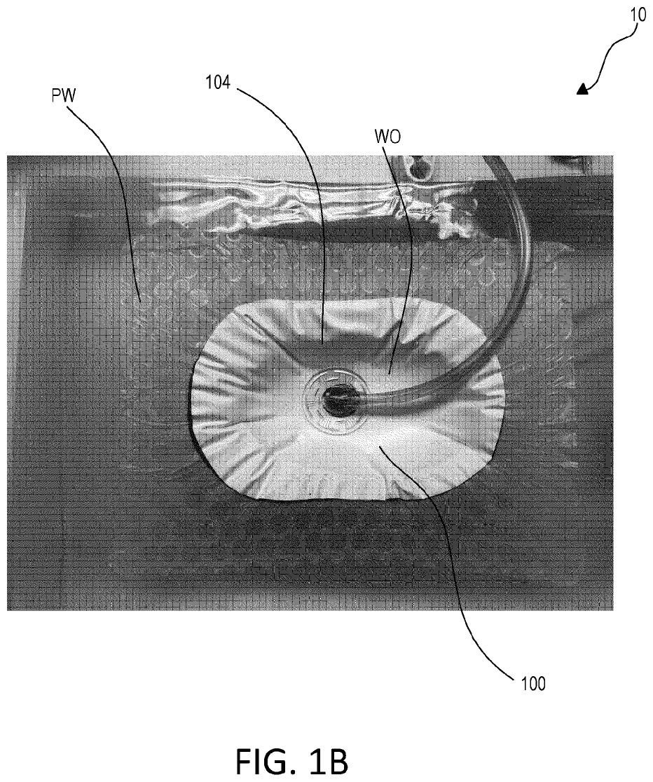

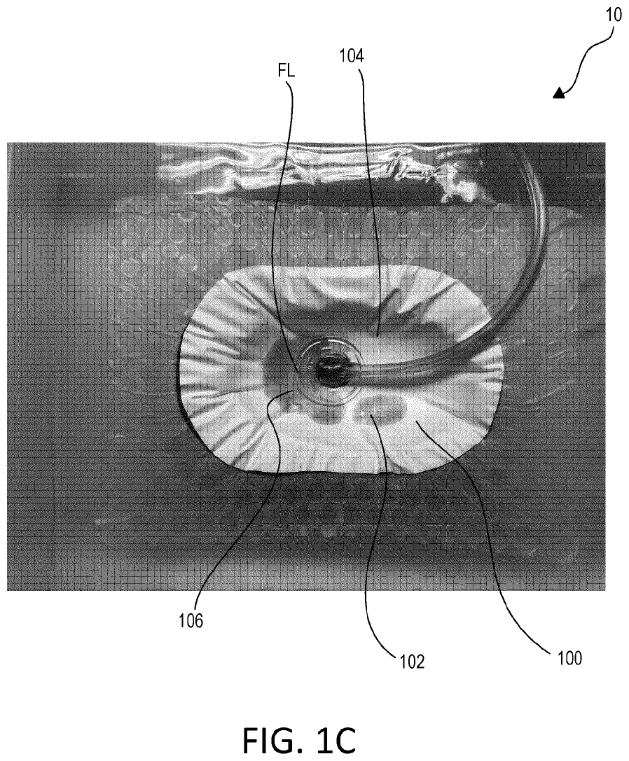

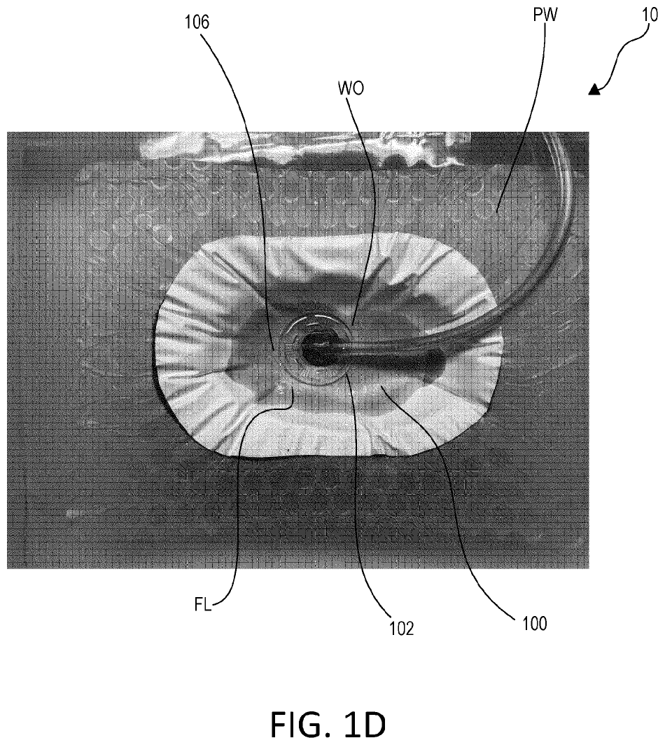

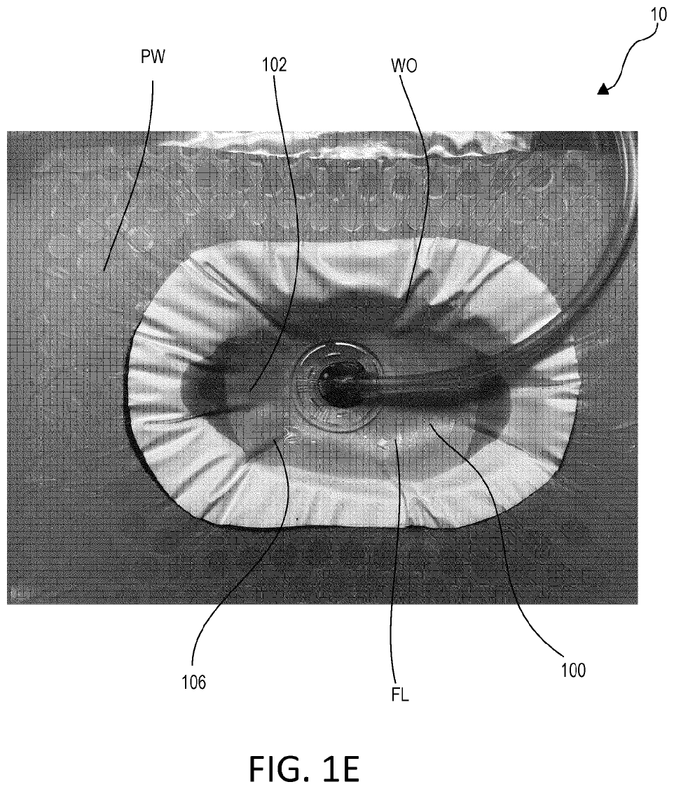

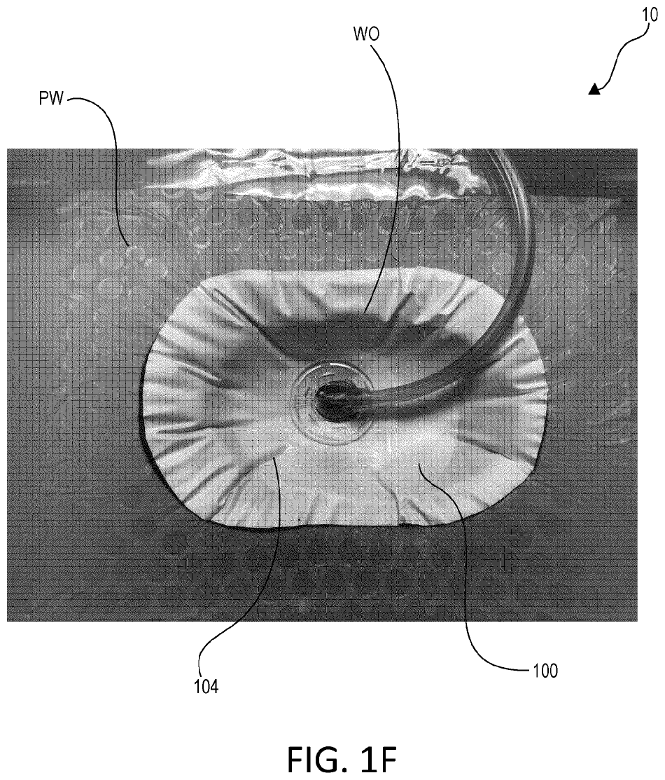

Referring to FIGS. 1A-1F, an exemplary wound therapy dressing in accordance with the principles of the present disclosure is described. As shown in FIG. 1A, wound therapy dressing 10 having at least one selectively transparent layer 100 is disclosed. Selectively transparent layer 100 may have a refractive index wherein interaction between selectively transparent layer 100 and fluid FL from a wound WO switches selectively transparent layer 100 from first, opaque state 104 to second, transparent state 106, as shown in FIG. 1C. Selectively transparent layer 100 may extend over both wound WO and periwound PW. Selectively transparent layer 100 further may have, be constituted of, or be coated with microporous particles.

FIG. 1B depicts an image of wound therapy dressing 10 at 125 mmHg prior to fluid FL delivery. FIG. 1C shows a top view of wound therapy dressing 10 wherein an initial fluid FL instillation is being delivered to the base of wound WO and selectively transparent layer 100 has begun to switch from first, opaque state 104 to second, transparent state 106. Fluid (FL) from a wound may include exudate or any secretion from a treatment site. The refractive index of selectively transparent layer 100 may be within a range that is substantially similar to that of the fluid (FL). In some embodiments, selectively transparent layer 100 may have a refractive index between about 1.0 and about 2.0. In some embodiments, selectively transparent layer 100 may have a refractive index between about 1.2 and about 1.75. In some embodiments, selectively transparent layer 100 may have a refractive index between about 1.33 and about 1.6.

Selectively transparent layer 100 further may comprise minerals. In some embodiments, exemplary minerals include, but are not limited to, silicas, silicates, fluorites, or fluorides. Selectively transparent layer 100 also may comprise polymers. Exemplary polymers include, but are not limited to, fluoropolymers, silicones, or acrylics.

FIG. 1D shows a top view of wound therapy dressing 10 where 60 mL of fluid FL has been delivered to dressing 10, and second, transparent state 106 is visible. Depending on the extent of fluid FL flow into wound WO and periwound PW, there may be one or more than one transparent region 102 in selectively transparent layer 100, without entire layer 100 being transparent. FIG. 1E depicts a top view of wound therapy dressing 10 where 120 mL of fluid FL has been delivered to both wound WO and periwound PW and is beginning to be withdrawn. Transparent region 102 is clearly visible and shows where fluid FL has spread throughout wound therapy dressing 10, creating second, transparent state 106. As illustrated in FIG. 1F, when selectively transparent layer 100 dries it returns to opaque state 104. In this embodiment, the image is taken at 1 hour after fluid delivery, however, the moisture vapor transmission rate of layer 100 can impact how quickly layer 100 returns to opaque state 104.

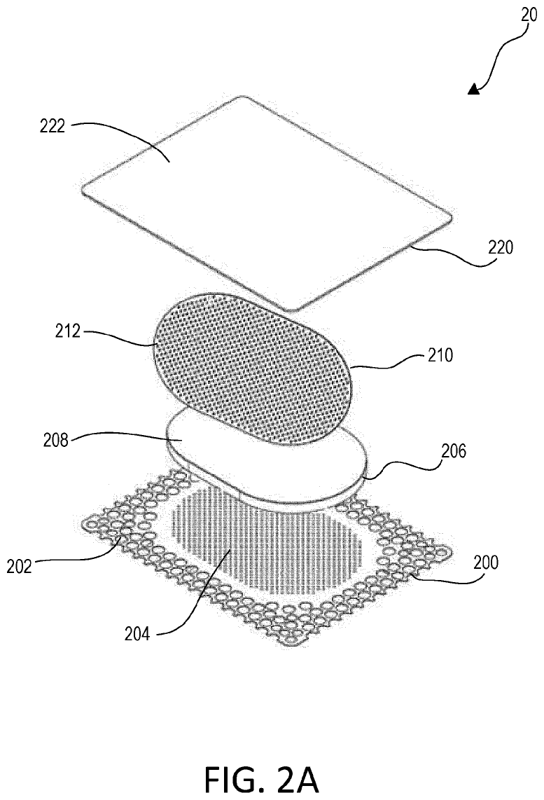

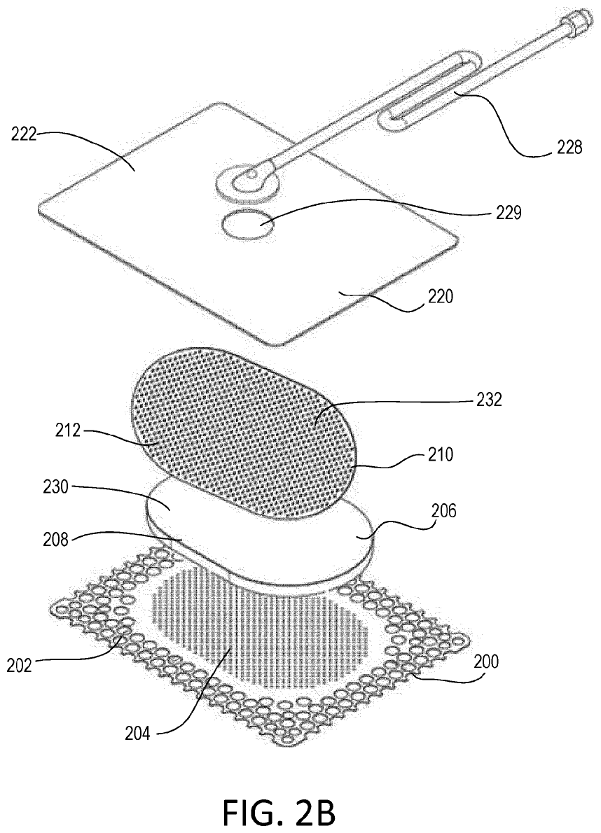

A wound therapy dressing having four layers configured to be assembled in a stacked relationship is also provided herein and shown in FIGS. 2-4. As shown in FIG. 2A, wound therapy dressing 20 may include: i) first layer 200 having polymer film 202 comprising a plurality of fluid restrictions 204, configured to be applied to treatment site TS; ii) second layer 206 which may include manifold 208; iii) third layer 210, which may include selectively transparent layer 212 that is opaque when dry in a first state and translucent when wet in a second state; and iv) fourth layer 220 comprising transparent polymer drape 222. The second, translucent state may occur at a point in time subsequent to the first, opaque state. Selectively transparent layer 212 becomes transparent when it interacts with fluid FL with a refractive index substantially similar to selectively transparent layer 212. Fluid FL may be an exudate or a secretion from a treatment site TS. In some embodiments, selectively transparent layer 212 may have a refractive index between about 1.0 and about 2.0. In some embodiments, selectively transparent layer 212 may have a refractive index between about 1.2 and about 1.75. In some embodiments, selectively transparent layer 212 may have a refractive index between about 1.33 and about 1.6.

First layer 200 further may be configured to expand in response to a pressure gradient across film 202. In some embodiments, third layer 210 may be laminated on top of second layer 206 and underneath fourth layer 220. Third layer 210 further may perforated; it may be perforated by a user or come perforated after manufacture. Fourth layer 220 may extend over and secure first layer 200, second layer 206, and third layer 210 at wound treatment site TS. As in dressing 10, discussed above, selectively transparent layer 212 returns to an opaque state as fluid FL evaporates. Further, first portion 224 of selectively transparent layer 212 may become translucent when exposed to fluid FL and remaining portion 226 of selectively transparent layer 212 not exposed to fluid FL may remain opaque.

Referring now to FIG. 2B, dressing 20 further may include negative pressure wound therapy input device 228. Negative pressure wound treatment input device 228 couples to hole 229 in drape layer 222 through which air is withdrawn from treatment site TS. Manifold 208 may be made of foam 230. Foam 230 further may be made of various polymers or plastics, including, but not limited to, polyurethane. Foam 230 may be porous and in some embodiments has an average pore size in a range of 400-600 microns. In some embodiments, manifold 208 may be a corrugated structure. Referring again to FIG. 2B, selectively transparent layer 212 also may be made of or coated with microporous particles 232. Selectively transparent layer 212 also may include or be made of minerals. Exemplary minerals include, but are not limited to, silicas, silicates, fluorites, or fluorides. Selectively transparent layer 212 also may comprise polymers. Exemplary polymers include, but are not limited to, fluoropolymers, silicones, or acrylics.

In one embodiment, drape 222 may be a clear adhesive before being coated with microporous particles. One or both of manifold 208 and selectively transparent layer 212 may be hydrochromic. Further, any hydrochromic material may be polyvinylidene difluoride (PVDF).

In some embodiments, selectively transparent layer 212 may be made of areas in an alternating radial pattern extending from a center to an outside edge of third layer 210, rather than a continuous area. Such pattern may be advantageous in monitoring the rate of progression of the fluid across the dressing.

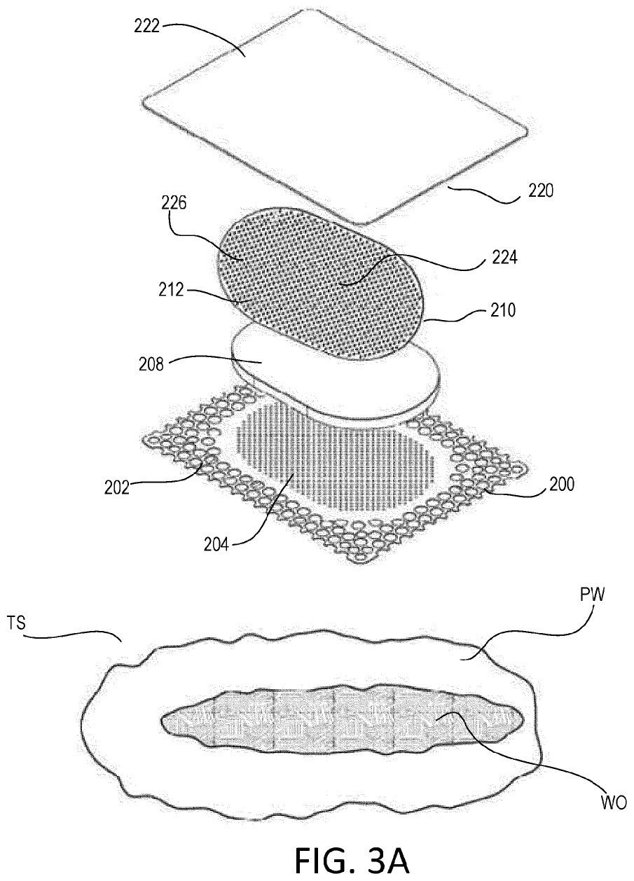

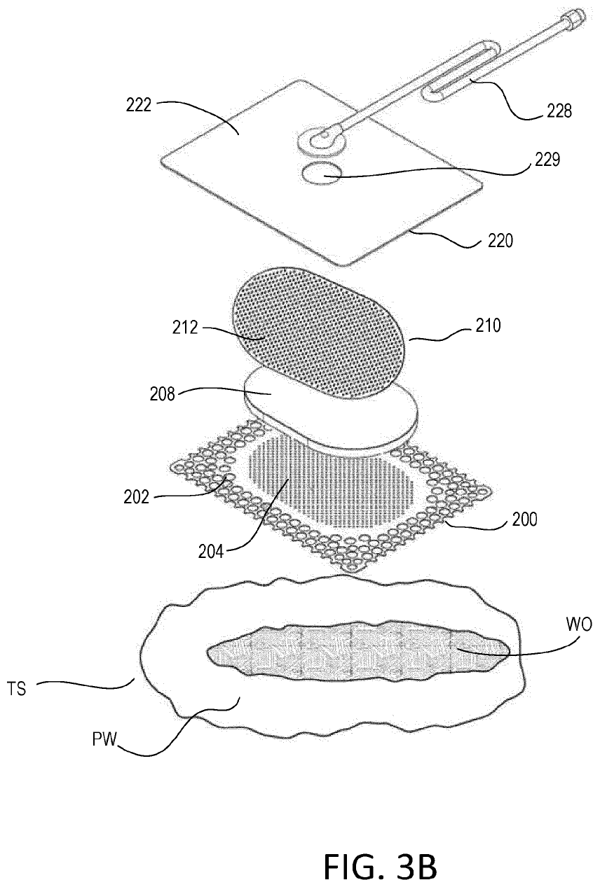

FIG. 3A depicts wound therapy dressing 20 in exploded view before it is assembled and in relation to treatment site TS. In some embodiments, wound therapy dressing 20 extends over both wound WO and periwound PW. FIG. 3B also depicts wound therapy dressing 20 in exploded view before it is assembled and applied to treatment site, however, FIG. 3B further depicts an embodiment that includes negative pressure wound treatment input device 228 and hole 229 incorporated with drape layer 222.

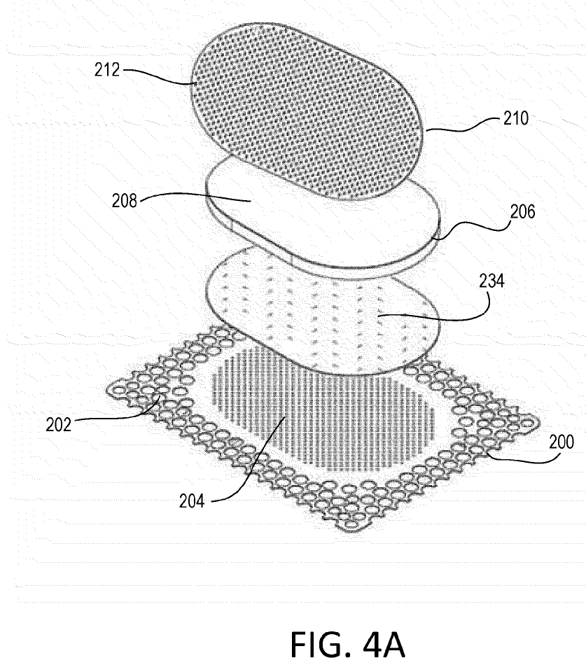

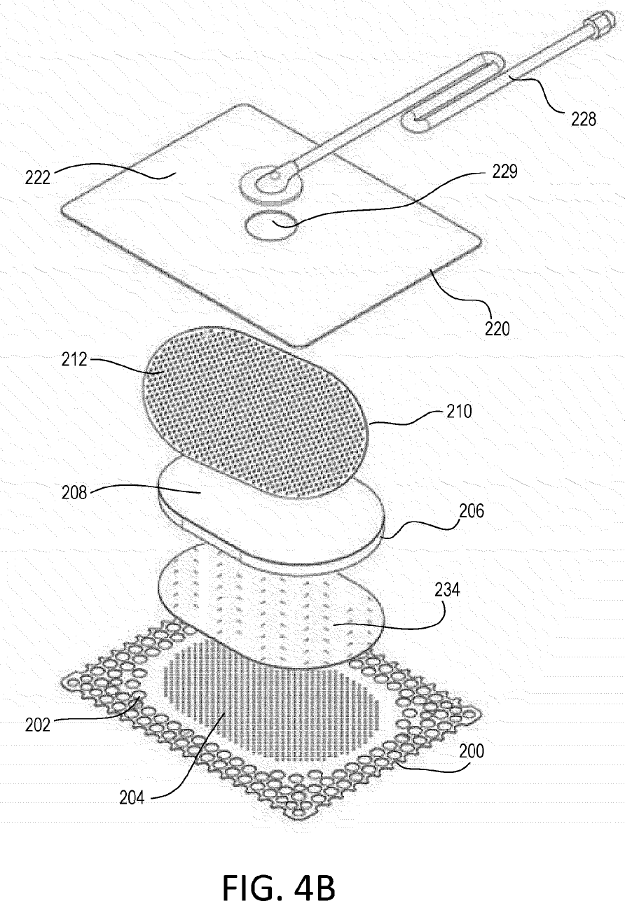

FIG. 4A depicts an alternative embodiment wherein wound therapy dressing 20 further includes hydrophobic polymer layer 234. In some embodiments, hydrophobic polymer layer 234 may be polyethylene film. The simple and inert structure of polyethylene can provide a surface that interacts little, if any, with biological tissues and fluids, providing a surface that may encourage the free flow of liquids and low adherence, which can be particularly advantageous for many applications. More polar films suitable for laminating to a polyethylene film include polyamide, co-polyesters, ionomers, and acrylics. To aid in the bond between a polyethylene and polar film, tie layers may be used, such as ethylene vinyl acetate, or modified polyurethanes. An ethyl methyl acrylate (EMA) film may also have suitable hydrophobic and welding properties for some configurations. FIG. 4B depicts an alternative embodiment wherein the wound therapy dressing further includes hydrophobic polymer layer 234 and negative pressure wound treatment input device 228.

### Method of Use:

A method of use for the wound therapy dressings described above is also disclosed herein. In one instance, the method includes i) applying to treatment site a wound therapy dressing comprising at least one selectively transparent layer with a refractive index wherein interaction between the selectively transparent layer and fluids from a treatment site switches the selectively transparent layer from a first, opaque state to a second, transparent state; (ii) sealing the dressing to epidermis adjacent or around the treatment site; (iii) observing the selectively transparent layer for at least one transparent region 102, wherein transparent region 102 on the selectively transparent layer indicates fluid in that area of the treatment site; and (iv) replacing the dressing when the transparent region 102 overtakes or extends over the periwound (PW) for a predetermined time period. The time period may be established to account for the MVTR of the dressing and the surrounding environment. In some instances, a relatively small amount of fluid may evaporate off in a relatively short time, such that dressing replacement is not necessary. In other instances, a large amount of fluid may persist after a relatively longer time period, such that dressing replacement may be appropriate in the discretion of a caregiver.

The method may be applied to extend the selectively transparent layer over both wound (WO) and periwound (PW). The appearance of the transparent region 102 in the periwound (PW) area indicates the presence of fluid which may prompt a caregiver to replace the dressing to avoid a possible maceration. The presence or absence of transparent region 102 also indicates the moisture vapor transmission rate of the dressing. One intention of the therapy is to prevent maceration of a periwound (PW). The dressings used in conjunction with the method may further include a negative pressure wound treatment input device 228. Negative pressure wound input or interface device 228 may couple to hole 229 in the drape layer through which air is withdrawn from the treatment site. Negative pressure input devices 228, used in conjunction with these methods, may apply reduced or excess pressure and is intended to promote healing and tissue granulation.

The dressings and methods described herein may provide significant advantages over prior dressings. For example, some dressings for negative-pressure therapy can require time and skill to be properly sized and applied to achieve a good fit and seal. In contrast, some embodiments of the dressing provide a negative-pressure dressing that is simple to apply, reducing the time to apply and remove. In some embodiments, for example, the dressing may be a fully integrated negative-pressure therapy dressing that can be applied to a tissue site (including on the periwound) in one step, without being cut to size, while still providing or improving many benefits of other negative-pressure therapy dressings that require sizing. Such benefits may include good manifolding, beneficial granulation, protection of the peripheral tissue from maceration, and a low trauma and high-seal bond. These characteristics may be particularly advantageous for surface wounds having moderate depth and medium-to-high levels of exudate. Some embodiments of the dressing may remain on the tissue site for at least 5 days, and some embodiments may remain for at least 7 days. Antimicrobial agents in the dressing may extend the usable life of the dressing by reducing or eliminating infection risks that may be associated with extended use, particularly use with infected or highly exuding wounds.

While various illustrative embodiments of the invention are described above, it will be apparent to one skilled in the art that various changes and modifications may be made herein without departing from the invention. The appended claims are intended to cover all such changes and modifications that fall within the scope of the invention.

1. A wound therapy dressing, comprising:
at least one selectively transparent layer comprising a refractive index wherein interaction between the selectively transparent layer and fluid from a wound switch the selectively transparent layer from a first, opaque state to a second, transparent state, and further wherein the selectively transparent layer is configured to extend over both a wound and a periwound.
wherein a presence of the fluid as represented by the transparent state indicates where the dressing wetted.

2. (canceled)

3. The dressing of claim 1, wherein the selectively transparent layer comprises microporous particles.

4. (canceled)

5. The dressing of claim 1, wherein the refractive index of the selectively transparent layer is within a range that is substantially similar to that of the fluid.

6. (canceled)

7. (canceled)

8. The dressing of claim 3, wherein the selectively transparent layer comprises a refractive index between about 1.33 and about 1.6.

9. The dressing of claim 3, wherein the selectively transparent layer comprises minerals, and the minerals comprise silicas, silicates, fluorites, or fluorides.

10. (canceled)

11. The dressing of claim 3, wherein the selectively transparent layer comprises polymers, and the polymers comprise fluoropolymers, silicones, or acrylics.

12. (canceled)

13. A wound therapy dressing, comprising:
a first layer comprising a polymer film comprising a plurality of fluid restrictions through the polymer film, wherein the first layer is configured to be applied to a treatment site;
a second layer comprising a manifold;
a third layer comprising a selectively transparent layer, wherein in a first state the selectively transparent layer is opaque when dry and in a second state the selectively transparent layer is translucent when wetted by a fluid with a refractive index substantially similar to the selectively transparent layer; and
a fourth layer comprising a substantially transparent polymer drape
wherein the layers are configured to be assembled in a stacked relationship.

14. (canceled)

15. (canceled)

16. (canceled)

17. (canceled)

18. The dressing of claim 13, wherein the selectively transparent layer comprises a refractive index between about 1.33 and about 1.6.

19. (canceled)

20. The dressing of claim 13, wherein the third layer is laminated on top of the second layer and underneath the fourth layer.

21. The dressing of claim 13, wherein the third layer is perforated.

22. The dressing of claim 13, wherein the fourth drape layer extends over and adheres the first layer, the second layer, and the third layer to a wound treatment site.

23. The dressing of claim 13, wherein the selectively transparent layer returns to the first, opaque state as fluid evaporates.

24. The dressing of claim 13, wherein a first portion of the selectively transparent layer becomes translucent when exposed to the fluid and a remaining portion of the selectively transparent layer not exposed to the fluid remains opaque.

25. The dressing of claim 13 further comprising a negative pressure wound therapy input device.

26. (canceled)

27. (canceled)

28. (canceled)

29. (canceled)

30. (canceled)

31. The dressing of claim 13, wherein the selectively transparent layer comprises microporous particles.

32. (canceled)

33. The dressing of claim 31, wherein the selectively transparent layer comprises minerals, and the minerals comprise silicas, silicates, fluorites, or fluorides.

34. (canceled)

35. The dressing of claim 31, wherein the selectively transparent layer comprises polymers, and the polymers comprise fluoropolymers, silicones, or acrylics.

36. (canceled)

37. (canceled)

38. (canceled)

39. (canceled)

40. The dressing of claim 13, wherein the selectively transparent layer indicates the presence or absence of fluids over the periwound.

41. The dressing of claim 13, wherein at least one of the manifold and the selectively transparent layer is hydrochromic.

42. (canceled)

43. The dressing of claim 41, wherein the hydrochromic layer comprises polyvinylidene difluoride (PVDF).

44. The dressing of claim 13, wherein the second state occurs at a point in time subsequent to the first state.

45. (canceled)

46. The dressing of claim 13, further comprising a hydrophobic polymer layer.

47. (canceled)

48. A method for wound therapy, comprising:
(i) applying to a treatment site a wound therapy dressing comprising at least one selectively transparent layer comprising a refractive index wherein interaction between the selectively transparent layer and fluids from a treatment site switches the selectively transparent layer from a first, opaque state to a second, transparent state;
(ii) sealing the dressing to epidermis adjacent or around the treatment site; and
(iii) observing the selectively transparent layer for at least one transparent region, wherein a transparent region on the selectively transparent layer indicates fluid in that area of the treatment site.

49. The method of claim 48, wherein the transparent region indicates the presence of fluid in a periwound region of the dressing.

50. (canceled)

51. (canceled)

52. (canceled)

53. The method of claim 49, wherein the dressing further comprises a negative pressure wound treatment input device.

54. (canceled)

55. (canceled)

56. The method of claim 48, further comprising:
(iv) replacing the dressing when the transparent region extends over the periwound for a predetermined time period.
